(19) United States
(12) Patent Application Publication
(10) Pub. No.: US 2021/0369232 A1
(43) Pub. Date: Dec. 2, 2021

(54) SYSTEMS AND METHODS FOR EVALUATING RESPIRATORY FUNCTION USING A SMARTPHONE

(71) Applicant: UNIVERSITY OF PITTSBURGH-OF THE COMMONWEALTH SYSTEM OF HIGHER EDUCATION, Pittsburgh, PA (US)

(72) Inventors: WEI CHEN, Wexford, PA (US); WEI GAO, Mars, PA (US); ERICK FORNO, Pittsburgh, PA (US)

(73) Assignee: UNIVERSITY OF PITTSBURGH-OF THE COMMONWEALTH SYSTEM OF HIGHER EDUCATION, Pittsburgh, PA (US)

(21) Appl. No.: 17/333,748

(22) Filed: May 28, 2021

Related U.S. Application Data
(60) Provisional application No. 63/031,876, filed on May 29, 2020.

(51) Int. Cl. A61B 7/00; A61B 5/00; A61B 5/08

(52) U.S. Cl. CPC A61B 7/003; A61B 5/6898; A61B 5/08; A61B 5/7475; A61B 5/7203; A61B 5/7225; A61B 5/7264

(57) ABSTRACT

A method of estimating a number of lung function indices of an individual. The method includes: transmitting an ultrasound signal toward a chest of the individual from a speaker of a smartphone while the individual is holding the smartphone in a hand of the individual; receiving in a microphone of the smartphone a reflected signal reflected from the chest of the individual in response to the ultrasound signal; extracting a number of features from the reflected signal; and providing the number of features to a neural network regression model, wherein the neural network regression model estimates the number of lung function indices based on the number of features and based on a non-linear correlation between chest wall motion and human lung function.

### CROSS REFERENCE TO RELATED APPLICATIONS

This application claims priority to U.S. provisional application No. 63/031,876 filed May 29, 2020, entitled “Systems and Methods for Evaluating Respiratory Function Using a Smartphone”, the contents of which are incorporated in its entirety herein by reference.

### STATEMENT OF GOVERNMENT SUPPORT

This invention was made with government support under grant #CNS-1812407 and grant #CNS-1812399 awarded by the National Science Foundation (NSF). The government has certain rights in the invention.

### TECHNICAL FIELD

The present invention relates to systems and methods for pulmonary function testing, and, in particular, to systems and methods for estimating lung function, calculating airway mechanics and/or detecting airway obstructions using an electronic apparatus, such as a smartphone or similar device.

### BACKGROUND OF THE INVENTION

Respiratory diseases, such as asthma, chronic pulmonary disease (COPD) and acute respiratory distress syndrome (ARDS), constitute a significant public health challenge. Over 260 million people worldwide have asthma, including 7% of children and 8% of adults in the United States. Similarly, COPD affects over 15 million people in the U.S., and was the third leading cause of death in the country in 2014. These diseases are characterized as various types of airway obstruction. Pulmonary function testing (PFT), as an objective assessment of airway obstruction, is hence crucial in disease evaluation and monitoring. For example, spirometry, the most commonly used PFT in clinic, measures the volume and velocity of breathing airflow. Spirometry can also be used to judge shortness of breath and airway inflammation, both of which are important symptoms of the coronavirus disease (COVID-19). Ideally, spirometry should be conducted frequently to timely detect and avoid frequent disease exacerbations that cause emergency department visits or hospitalizations. However, current spirometers are generally bulky, difficult to operate, expensive, or require regular calibration, which limits their use to the clinic and precludes daily home use. Recent efforts have reduced the size of spirometers, but their costs (>$2,000) are still too high for home use. Low-cost spirometers priced at <$100, on the other hand, have low accuracy and could produce >20% error.

### SUMMARY OF THE INVENTION

Embodiments of the present invention utilize technology that is commonly available in smartphones and similar electronic devices to produce accurate, convenient, yet low-cost and unobtrusive, pulmonary function measurements by using the device's speaker and microphone generally as an ultrasonic “sonar”. The technology is non-invasive and easy to implement using equipment that is generally already present in such electronic devices. In addition to lung function, the technology may also aid in 1) detection of abnormal breathing patterns, such as irregular respiratory rate, 2) analysis of lung sounds (e.g., wheezing and crackles) where auscultation by a trained provider is not feasible, and 3) detection of upper and lower airway narrowing or obstruction (e.g., foreign bodies, vocal cord dysfunction, or tracheo/laryngomalacia).

As one aspect of the invention, a method of estimating a number of lung function indices of an individual is provided. The method comprises: transmitting an ultrasound signal toward a chest of the individual from a speaker of a smartphone while the individual is holding the smartphone in a hand of the individual; receiving in a microphone of the smartphone a reflected signal reflected from the chest of the individual in response to the ultrasound signal; extracting a number of features from the reflected signal; and providing the number of features to a neural network regression model, wherein the neural network regression model estimates the number of lung function indices based on the number of features and based on a non-linear correlation between chest wall motion and human lung function.

The number of features may be extracted from a number of exhalation portions identified from the reflected signal.

Extracting the number of features from the reflected signal may comprise: converting the reflected signal to a number of I/Q traces on a complex plane to quantify an impact of random motion of the smartphone on the reflected signal; and adaptively removing the impact by correcting for distortion caused by the random motion and calibrating the reflected signal as if it was produced by a stationary device.

As a further aspect of the present invention, an apparatus for estimating a number of lung function indices of an individual is provided. The apparatus comprises: a speaker; a microphone; and a controller coupled to the speaker and the microphone and structured and configured for: transmitting an ultrasound signal toward a chest of the individual from the speaker while the individual is holding the apparatus in a hand of the individual; receiving in the microphone a reflected signal reflected from the chest of the individual in response to the ultrasound signal; extracting a number of features from the reflected signal; and providing the number of features to a neural network regression model implemented in the apparatus, wherein the neural network regression model estimates the number of lung function indices based on the number of features and a non-linear correlation between chest wall motion and human lung function.

The number of features may be extracted from a number of exhalation portions identified from the reflected signal.

Extracting the number of features from the reflected signal may comprise: converting the reflected signal to a number of I/Q traces on a complex plane to quantify an impact of random motion of the smartphone on the reflected signal, and adaptively removing the impact by correcting for distortion caused by the random motion and calibrating the reflected signal as if it was produced by a stationary device.

The apparatus may be a smartphone.

As yet another aspect of the present invention, a method of calculating airway mechanics and/or detecting airway obstructions in an individual is provided. The method comprises: directing an ultrasound signal generated by a smartphone into the airways of the individual through an interface attached to the smartphone and coupled to a speaker of the smartphone, wherein the interface has a mouthpiece structured to be received in a mouth of the individual; receiving in a microphone of the smartphone and through the interface a reflected signal reflected from the airways of the individual in response to the ultrasound signal; filtering and denoising the reflected signal to produce an adjusted reflected signal; and isolating a mixture of signals in the adjusted reflected signal and calculating from the isolated mixture of signals the airway mechanics and/or detecting from the isolated mixture of signals the airway obstructions.

As another aspect of the present invention, a non-transitory computer readable medium storing one or more programs, including instructions, which when executed by a processor, causes the processor to perform any of the aforementioned methods is provided.

A yet a further aspect of the present invention an apparatus for calculating airway mechanics and/or detecting airway obstructions in an individual is provided. The apparatus comprises: a speaker; a microphone; an interface sized and structured to convey an ultrasound signal between the speaker and the microphone and a mouth of the individual; and a controller coupled to the speaker and the microphone and structured and configured for: transmitting an ultrasound signal from the speaker into the airways of the individual through the interface; receiving from the in the microphone a reflected signal from the interface reflected from the airways of the individual in response to the ultrasound signal; filtering and denoising the reflected signal to produce an adjusted reflected signal; and isolating a mixture of signals in the adjusted reflected signal and calculating from the isolated mixture of signals the airway mechanics and/or detecting from the isolated mixture of signals the airway obstructions.

The apparatus may be a smartphone.

The interface may comprise: an interface tube having a first end and a second end opposite the first end, the second end being sized and configured to be placed in the mouth of the individual; and an adaptor having a first portion selectively engaged on and around an end of the smartphone so as to encompass the speaker and the microphone and a second portion coupled to the first end of the interface tube.

The interface tube may further include a stopper portion structured to position the second end of the interface tube a predetermined distance from a front tooth of the individual.

The interface tube may include an opening defined therein between the first end and the second end thereof.

The adaptor may comprise one or more of an auxiliary speaker and/or an auxiliary microphone in communication with the controller.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

A full understanding of the invention can be gained from the following description of the preferred embodiments when read in conjunction with the accompanying drawings in which:

FIG. 1 is a schematic diagram of an electronic apparatus in accordance with one example embodiment of the present invention which is employed in carrying out methods in accordance with example embodiments of the present invention.

FIG. 2 is a schematic representation showing general details of a lung function (e.g., mobile spirometry) test being carried out on a patient in accordance with an example embodiment of the present invention using the apparatus of FIG. 1.

FIG. 3 is an example flow-volume loop graph created from spirometry measurements that depicts the correlation between the volume and velocity of airflow during inhalation and exhalation by a patient.

FIG. 4A is a schematic illustration of a sectional view of a chest cavity showing chest wall motion between full inhalation and after exhalation by a patient.

FIG. 4B is a graph showing the correlation between chest wall position and lung volume,

FIG. 5A is a view of a patient holding an apparatus such as shown in FIG. 1 in position for carrying out a spirometry test in accordance with an example embodiment of the present invention.

FIG. 5B is a general overview of the operation of an apparatus such as shown in FIG. 1 in carrying out a spirometry test in accordance with an example embodiment of the present invention.

FIG. 6A is an I/Q trace of a signal received by an apparatus such as shown in FIG. 1 during a spirometry test when the apparatus is stationary.

FIG. 6B is an I/Q trace of a signal received by an apparatus such as shown in FIG. 1 during a spirometry test when the apparatus is hand-held and random movements of the apparatus occur.

FIG. 7 is a graph illustrating the impact of body movements during a spirometry test on the measured chest wall displacement.

FIG. 8 is a graph showing measured chest wall displacement and speed and identifying features of chest wall motion used to estimate lung function indices.

FIG. 9 shows the outcomes from a study in which both spirometry tests using an apparatus and method in accordance with an example embodiment of the present invention and clinical grade spirometers simultaneously being used as the inputs and outputs, respectively, to train a neural network in accordance with an example embodiment of the present invention.

FIG. 10 shows graphs illustrating examples of a patient exhaling with insufficient power and failing to keep an upright posture.

FIGS. 11A-11C show example I/Q traces and details of approaches for correcting/addressing distortions thereof in accordance with example embodiments of the present invention.

FIG. 12A is an arrangement for evaluating the approaches discussed in regard to FIGS. 11A-11C.

FIG. 12B is a graph showing results obtained using the arrangement of FIG. 12A.

FIGS. 13A-13C are graphs of results of testing carried out to verify aspects of embodiments of the present invention.

FIG. 14 is a graph showing potential values for use in an example embodiment of the present invention.

FIGS. 15A-15C show example interactive displays of an application carrying out a method in accordance with an example embodiment of the present invention using an apparatus such as shown in FIG. 1.

FIG. 16 is an arrangement of a patient communication interface in accordance with an example embodiment of the present invention shown positioned on an example embodiment of an interface such as shown in FIG. 1.

FIG. 17 is an enlarged view of a portion of the arrangement of FIG. 16 showing details of a portion of the patient communication interface.

### DETAILED DESCRIPTION OF EXEMPLARY EMBODIMENTS

As used herein, the singular form of “a”, “an”, and “the” include plural references unless the context clearly dictates otherwise.

As used herein, the statement that two or more parts or components are “coupled” shall mean that the parts are joined or operate together either directly or indirectly, i.e., through one or more intermediate parts or components, so long as a link occurs.

As used herein, “directly coupled” shall mean that two elements are directly in contact with each other.

As used herein, the term “number” shall mean one or an integer greater than one (i.e., a plurality).

As used herein, the term “connected” shall mean that elements are electrically connected such that signals may pass from one of the elements to the other.

As used herein, the term “controller” shall mean a programmable analog and/or digital device (including an associated memory part or portion) that can store, retrieve, execute and process data (e.g., software routines and/or information used by such routines), including, without limitation, a field programmable gate array (FPGA), a complex programmable logic device (CPLD), a programmable system on a chip (PSOC), an application specific integrated circuit (ASIC), a microprocessor, a microcontroller, a programmable logic controller, or any other suitable processing device or apparatus. The memory portion can be any one or more of a variety of types of internal and/or external storage media such as, without limitation, RAM, ROM, EPROM(s), EEPROM(s), FLASH, and the like that provide a storage register, i.e., a non-transitory machine readable medium, for data and program code storage such as in the fashion of an internal storage area of a computer, and can be volatile memory or nonvolatile memory.

As used herein, the terms “component” and “system” are intended to refer to a computer related entity, either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution, and a component can be localized on one computer and/or distributed between two or more computers. While certain ways of displaying information to users are shown and described with respect to certain figures or graphs as screenshots, those skilled in the relevant art will recognize that various other alternatives can be employed.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

The disclosed concept will now be described, for purposes of explanation, in connection with numerous specific details in order to provide a thorough understanding of the subject invention. It will be evident, however, that the disclosed concept can be practiced without these specific details without departing from the spirit and scope of this innovation.

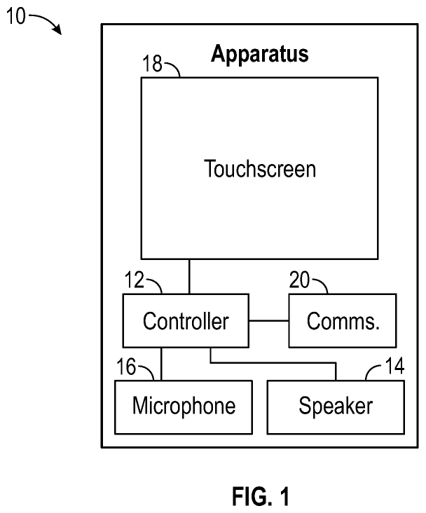

Embodiments of the present invention utilize an electronic apparatus 10 such as shown schematically in FIG. 1. Apparatus 10 includes a controller 12, a speaker 14, and a microphone 16. Controller 12 is connected to, and in communication with, both of speaker 14 and microphone 16. Controller 12 is structured and configured to cause speaker 14 to emit soundwaves of predetermined frequencies and to receive information regarding soundwaves, such as those produced by speaker 14, via microphone 16. Additionally, apparatus 10 may include one or more input and/or output devices connected to, and in communication with, controller 12, for example, without limitation, a touchscreen 18 such as shown in FIG. 1, and/or one or more communication arrangement(s) 20 in communication with controller 12 for providing input to, or communicating output from, controller 12. Such communication arrangement(s) may include, for example, without limitation, arrangements providing for wireless (e.g., Bluetooth, cellular, etc.), and/or wired communications to/from apparatus 10 and controller 12 thereof. In the example embodiments illustrated herein, electronic apparatus 10 is in the form of a commodity smartphone device (e.g., without limitation, an Android or iPhone device) such as readily available to the general masses. It is to be appreciated, however, that apparatus 10 may be other suitable electronic devices without varying from the scope of the present invention.

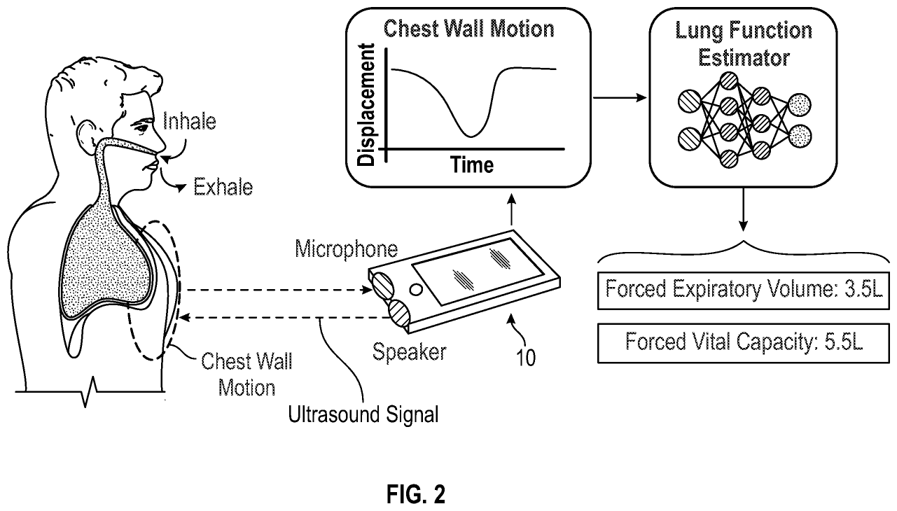

Referring now to FIG. 2, a first arrangement is shown in which apparatus 10, shown as a smartphone device, is employed to support complete, accurate and reliable spirometry tests out of clinic, with various environmental and human factors. Apparatus 10 utilizes the close correlation between lung function and chest wall motion of humans, a correlation that has been widely validated in clinical practice. Apparatus 10 measures chest wall motion of the patient, an externally observable biomarker, and interprets such motion into lung function indices. To measure such motion, speaker 14 of apparatus 10 is used to transmit ultrasound signals toward the patient. The signal reflected by the patient's chest wall is received by microphone 16 of apparatus 10 and subsequently analyzed by controller 12 of apparatus 10. In this way, the measurements carried out by apparatus 10 are 100% contactless and non-intrusive.

In order to ensure accurate spirometry tests, the error of measuring the chest wall motion by apparatus 10 should be at most a few millimeters. Current acoustic sensing systems achieve such accuracy using an apparatus that is always stationary, when monitoring humans' breath in sleep or tracking small targets' motions (e.g., human fingers). In contrast to such approaches, embodiments of the present invention enable daily spirometry tests anytime and anywhere by a user (i.e., the patient) holding apparatus 10 in one or both of their hands. When being hand-held, the received ultrasound signal can be easily affected by random motions of the apparatus 10. As discussed in detail below, to identify the impact of these random motions, the received signal from microphone 16 is converted by controller 12 to I/Q traces on the complex plane, so as to quantify such impact as the geometric distortions of I/Q trace. Then, such impact is adaptively removed by correcting such distortions and calibrating the received signal as if it was produced with a stationary apparatus.

A major challenge of interpreting chest wall motion into lung function indices, on the other hand, is the heterogeneous human factors that may impair the data quality in spirometry tests. For example, patients may fail to follow the spirometry protocol when using apparatus 10 without guidance from clinicians. To eliminate the impact of these human factors, apparatus 10 avoids estimating lung function indices directly from chest wall motion. Instead, specific features from the chest wall motion are extracted and used as the input to a neural network regression model provided as a portion of, or as a separate component in communication with, controller 12. In particular, these motion features are extracted only from the exhalation stage of spirometry, and multiple criteria are applied to ensure that such exhalation stage can be appropriately identified. From the descriptions provided herein, it is to be appreciated that apparatus 10 provides a convenient yet cost-free tool for continuous tracking and evaluation of pulmonary diseases, which are crucial to patients' wellbeing. Apparatus 10 can also contribute to early-stage diagnosis of COVID-19 out of clinic, and help reduce the burden on public healthcare systems in a pandemic. Some key characteristics of apparatus 10 are as follows:
Apparatus 10 is accurate. Its error of measuring chest wall motion is constrained within 4 mm. When being evaluated among healthy humans, its error of lung function monitoring is always lower than 3%.
Apparatus 10 is adaptive. It can precisely remove the impact from various environmental and human factors, and allows flexible variations of its position (up to 20 cm) and orientation (up to 30° tilting) during spirometry tests without impairing the accuracy. It also well adapts to humans' body conditions, as well as different types of clothes being worn.
Apparatus 10 is lightweight. It is contactless and does not require any extra hardware. When embodied as a smartphone, <15% of the smartphone's battery life is consumed with 1-hour of usage as described herein.
Apparatus 10 is easy to use. The programming utilized by controller 12 may be implemented as an Android or iPhone app, with the spirometry tests fully automated, thus requiring minimum involvement from users.

Having thus provided a general overview of a first arrangement of the present invention, a more detailed description of the steps carried out by controller 12 in performing methods in accordance with embodiments of the present invention, as well as the overall functionality of apparatus 10 will now be provided. As an initial matter, the clinical background of spirometry will be introduced, and the correlation between lung function and chest wall positioning/movement that is utilized by the present invention will be explained.

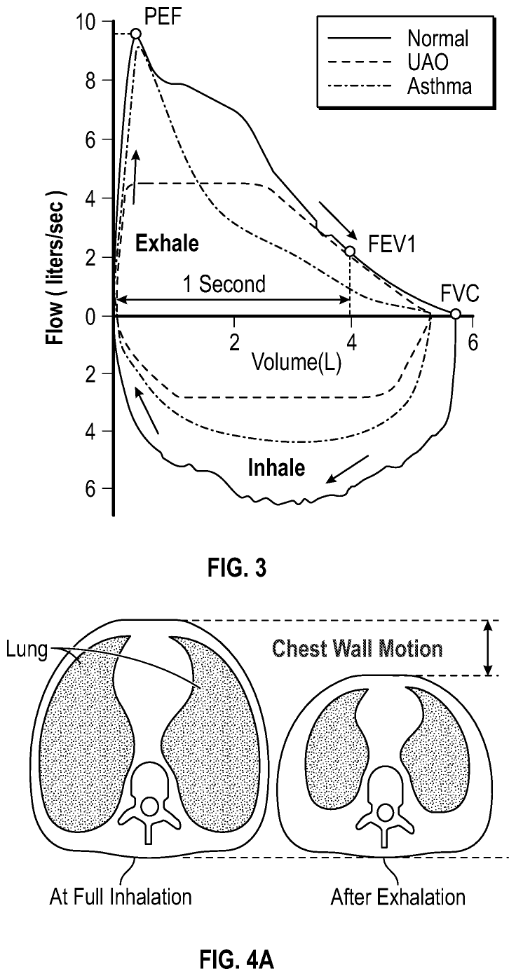

As is known, spirometry measures how fast and how much air a patient can breathe out. Before a test starts, the patient exhales all air from their lungs. A spirometry test consists of two stages: 1) the patient first takes a full inhalation and then 2) exhales as hard and as fast as possible, until no more air can be exhaled. As shown in FIG. 3, measurements from spirometry testing are represented by a flow-volume loop graph that depicts the correlation between the volume and velocity of airflow during inhalation and exhalation of a patient. As is known, the graphs of patients with pulmonary diseases are significantly different from those of healthy people. For example, patients with upper airway obstruction (UAO) exhibit apparent plateaus in the graph, and asthma patients exhibit “scooped” curves in the exhaling part of the graph. In practice, clinicians usually extract certain features from such graphs as lung function indices, for more convenient disease evaluation and monitoring. These indices include:
Peak expiratory flow (PEE)—is the maximum airflow velocity in exhalation. The average PEF of healthy males and females is around 10 L/sec and 8 L/sec, respectively. The PEF of asthma patients is as low as 5 L/sec.
Forced expiratory volume in 1 second (FEV1)—is the exhaled air volume in the first second of exhalation, and indicates the airway's resistance against breath. There are different sets of reference equations and normative values for FEV1, and FEV1 is decreased in patients with asthma, COPD, cystic fibrosis (CF), and other lung diseases. Decline of FEY 1 indicates disease deterioration in CF.
Forced vital capacity (FVC)—is the total air volume exhaled. Decline of FVC indicates disease deterioration in COPD patients.
FEV1/FVC—is the ratio of FEV1 and FVC. This ratio should be >80% among healthy people, but can be as low as 50-60% among asthma patients. Decline of FEV1/FVC can indicate deterioration of several lung disorders including asthma and CF.

In clinical practice, PEF measurements are highly variable, and clinicians mainly use the other three indices to evaluate lung function. To ensure accuracy, a patient usually completes multiple (e.g., 3-8) spirometry tests, and the maximum difference of FEV1 and FVC readings in these tests should be <0.15 L. In this way, measurement error of in-clinic spirometry is around 5%, which is the baseline to evaluate the performance of apparatus 10.

Since lung function greatly varies from one patient to another, the raw values of lung function indices are seldom used in clinic. Instead, clinicians usually categorize patients into subgroups according to their demographics (e.g., age, gender, race, etc.), and then convert the raw values of lung function indices into percentiles (% pred) over healthy people's data in the subgroup, provided by Global Lung Function Initiative (GLI). A percentile lower than 70% can indicate the presence of COPD or other lung diseases. Changes in the percentile of FEV1 or other indices can also indicate the presence of asthma or other lung diseases. Such percentiles are used by embodiment of the present invention as indicators to measure human lung function.

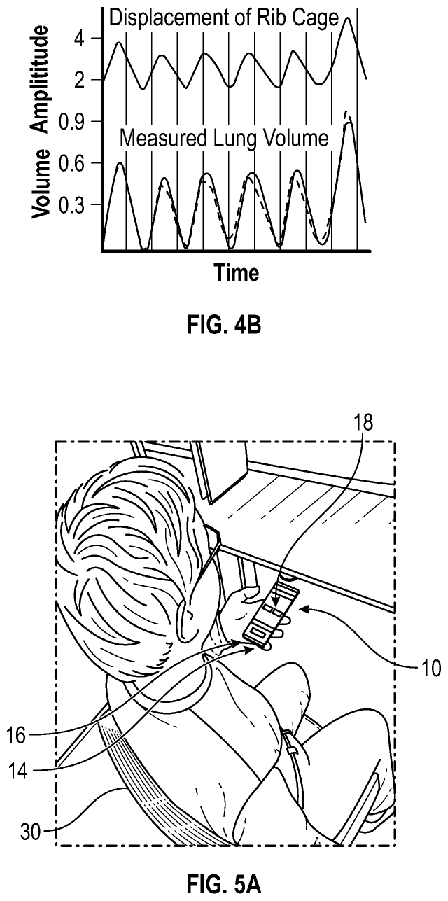

Correlation between lung function and chest wall motion has been clinically validated. Such correlation, as generally shown in FIG. 4A, originates from humans' ribcage expansion and contraction when breathing. These ribcage movements, when measured by a pneumotrace chest band, are consistent with the fluctuation of lung volume as shown in FIG. 4B. Clinical studies have also showed that humans' lung volume is proportional to the ribcage motion. Such correlation is utilized by embodiments of the present invention to measure the volume of breathing airflow externally, through the displacement of the chest wall in spirometry tests. Similarly, the velocity of airflow can be measured from the speed of chest motion. In particular, clinical studies have shown that asthma and COPD patients have significantly reduced chest wall motion, due to the abnormal changes of chest dimensions and the subsequent lateral ribcage indrawing. For example, patients and healthy people could have 20 mm mean difference on the ribcage anteroposterior motion, as well as 10 mm mean difference on the upper lateral motion. Such correlation has also been clinically validated to be significant and consistent across different human groups, such as different age groups, males and females with different chest structures and conditions, obese people with high BMI, etc. Hence, by measuring the chest wall motion of a patient as described herein, apparatus 10 can serve as a useful tool for pulmonary disease evaluation and tracking out of clinic.

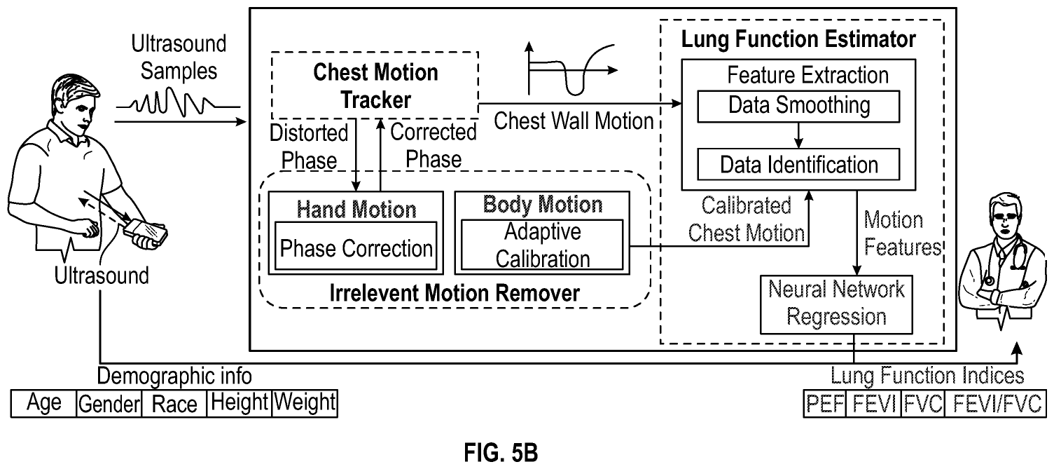

As shown in FIG. 5A, to carry out a spirometry test using apparatus 10 (shown as a smartphone), the patient hand-holds the smartphone and points the phone's bottom speaker and microphone to their chest. Then, the patient follows the spirometry protocol to inhale and exhale, which in the example of FIG. 5A is provided to the patient as instructions on touchscreen 18 of apparatus 10. For best results, such monitoring would be carried out with the patient's chest exposed (i.e., no shirt), however, testing of patients wearing various types of shirts from tight to loose fitting cotton t-shirts as well as other typical indoor clothing (i.e., not sweaters, jackets, etc.) has found that the impact of such indoor clothing is relative errors of less than 3%. Hence, spirometry tests using apparatus 10 may readily be carried out by patients wearing typical indoor clothing as well.

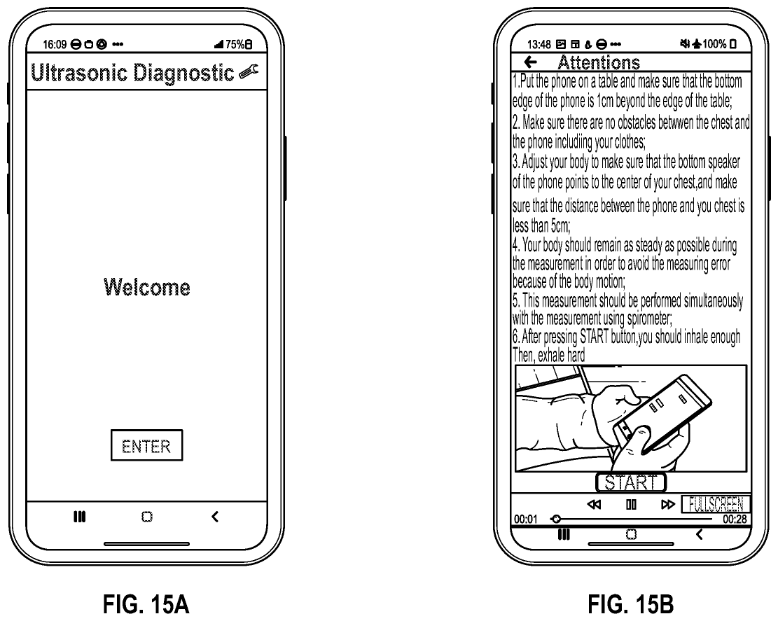

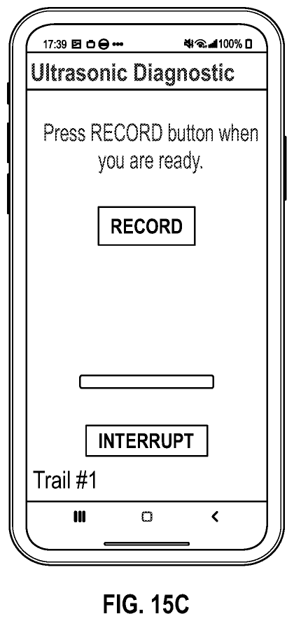

Some examples of displays provided via touchscreen 18 are discussed further below in conjunction with FIGS. 15A-15C. Being similar with in-clinic spirometry, the patient should maintain an upright posture by leaning their back against a fixed member such as a chair backrest 30 or other suitable member (e.g., a wall). In this way, the body trunk remains steady during the spirometry test, and the measured chest wall motion is only caused by inhalation and exhalation. Apparatus 10 tracks the patient's chest wall motion in both inhalation and exhalation stages of spirometry tests. To ensure accuracy, such chest wall motion is first examined and corrected to remove any impact of irrelevant motions. Afterwards, the corrected chest motion is used as the input to a neural network regression, which computes lung function indices based on the nonlinear correlation between chest wall motion and human lung function. These lung function indices are converted into % pred values based on the patient's demographic information, and then reported to pulmonary doctors (e.g., via communications arrangement 20 of apparatus 10) for remote disease evaluation and monitoring. A general overview of the operation of apparatus 10 in carrying out such a lung function test is shown in FIG. 5B.

Since humans' chest wall displacement in spirometry is usually lower than 60 mm, the error of chest motion tracking should be at most 3-4 millimeters, so that the error of lung function estimation is within 5%. To achieve such accuracy, apparatus 10 measures the phase change between the transmitted and received ultrasound signal. Considering the transmitted signal as Acos(2πfd(t)/c, the phase of the received signal, after being reflected by the chest wall, is:

φ(t)=4πfd(t)/c,

where c is sound speed, f is the frequency of the transmitted signal, and d(t) is the distance between chest wall and apparatus 10 at time t. When the chest wall moves during a time period [t₀, t₁], its displacement during this period is:

Δd=d(t₁)−d(t₀)=−c/(4πf)(φ(t1)−φ(t0))

When the ultrasound signal's frequency ranges between 17 kHz and 24 kHz, a 2 mm displacement causes the signal path length to change by 4 mm and corresponds to a phase change between 0.4π and 0.56π, large enough to be detected. Such detectability also allows for the use of multiple signals with different frequencies in this range to further improve the tracking accuracy.

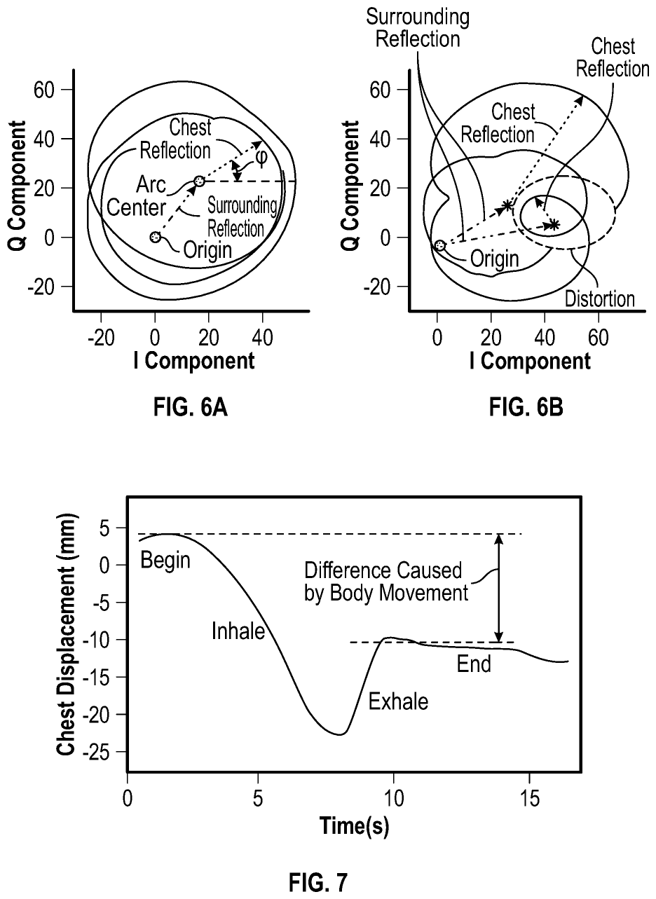

Since the chest wall's motion is measured as its relative displacement from apparatus 10, it could be easily affected by random movements of apparatus 10 (e.g., the patient cannot keep the hand-held apparatus 10 to be 100% stationary, the patient's body may unconsciously lean forward or backward when exhaling hard). To remove such irrelevant movements from the measured chest wall motion, an intuitive method in embodiments in which apparatus 10 is embodied as a smartphone is to measure the smartphone's movements using the built-in accelerometers of the smartphone. However, such approach is inaccurate due to the error accumulation when converting accelerometer readings into displacement via double integration. Instead, controller 12 investigates the abnormal characteristics from the measured chest wall motion itself. As shown in FIG. 6A, if apparatus 10 is stationary, the I/Q trace of the received signal should be a regular collection of concentric arcs. Otherwise, such I/Q trace produced from a hand-held apparatus 10 with random movements will be arbitrarily distorted. The reason, as shown in FIG. 6B, is that the received signal always contains reflections from both the chest wall and surrounding objects. When apparatus 10 is hand-held and randomly moving, the surrounding reflection varies and distorts the cumulatively received signal. Details of correcting such distortion are described further below.

Similarly, according to the spirometry protocol previously discussed, the patient's chest wall should be at the same position before and after a spirometry test, if the patient's body does not move during the test. The impact of body movements on the measured chest wall motion, as shown in FIG. 7, can hence be identified as the difference of chest wall position before and after a spirometry test. Details about removing such body movements are discussed below.

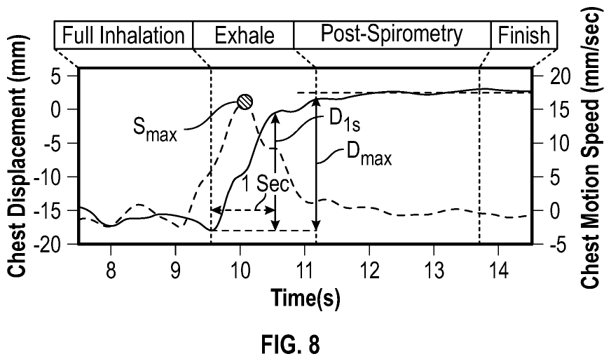

As shown in FIG. 8, controller 12 uses the following features of chest wall motion during the exhalation stage in spirometry to estimate lung function indices:
The maximum speed of chest wall motion (Sₘₐₓ), which corresponds to PEF.
The chest wall displacement in the first second of exhalation (D₁ₛ), which corresponds to FEV1.
The maximum chest wall displacement (Dₘₐₓ), which corresponds to FVC.

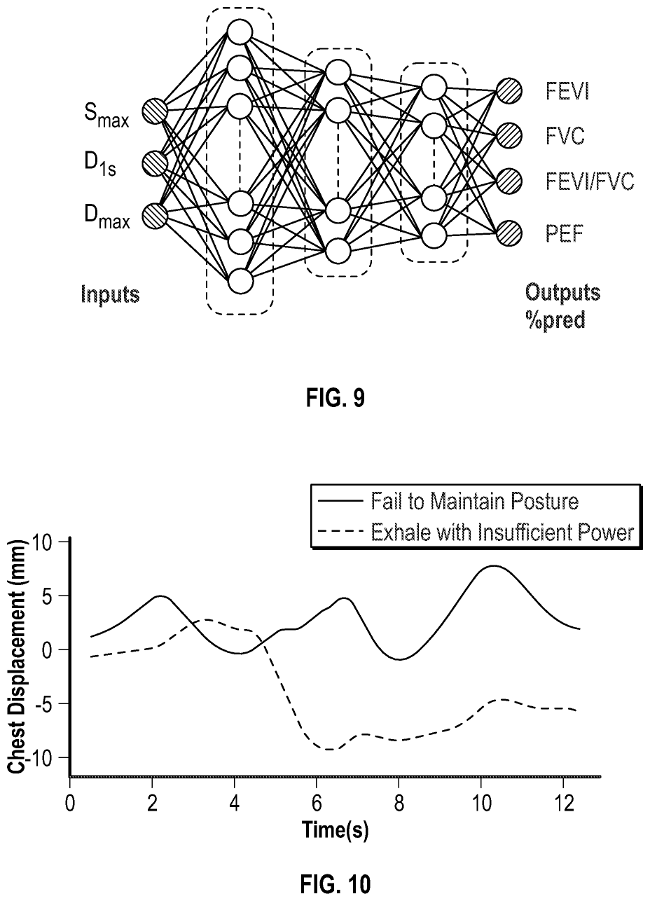

Controller 12 quantifies such correlation using a neural network regression model, which is built with data from clinical studies. Since such clinical data from patients is usually low in volume and could hence result in overfitting when training the model, in one example embodiment a Bayesian regularized neural network, which has good capability of generalization that avoids overfitting, has been utilized. In a clinical study, patients performed spirometry tests using apparatus 10 and clinical-grade spirometers at the same time. As shown in FIG. 9, the outcomes from the motion tracking and spirometers' measurements of apparatus 10 are then used as the inputs and outputs, respectively, to train the neural network. Afterwards, the trained neural network is loaded to the controller 12 of the patient's apparatus 10 (e.g., the patient's smartphone) for out-of-clinic use.

Both the accuracy and overhead of such inference depend on the complexity of the neural network. A neural network with more hidden layers and numbers of neurons improves the inference accuracy, but also increases its computation overhead. In an example embodiment of apparatus 10, three hidden layers are used empirically in the neural network associated with controller 12, we then balance between these two aspects by tuning the numbers of neurons in each layer. In general, it is ensured that the numbers of neurons in different hidden layers decrease as the network becomes deeper, and details of such tuning are described further below. Another challenge of such lung function estimation is the heterogeneous human factors, which may impair the data quality in spirometry tests and make it difficult to correctly identify the exhalation stage. Ideally, the measured chest wall motion, as shown in FIG. 8, should exhibit a sole rapid change of chest displacement of at least 10-15 mm, as a result of hard exhalation. However in practice, patients may not fully follow the spirometry protocol, due to lack of clinician's guidance or weak body conditions. As shown in FIG. 10, the patient may exhale with insufficient power and result in inadequate chest displacement; or the patient may fail to keep the upright posture and produce abnormal chest motions. Details about identifying the exhalation stage in each of these cases are provided further below.

Removal of irrelevant smartphone motions and patient body motions from the measured chest wall motion will now be discussed. As shown in FIG. 6B, motions of a hand-held apparatus 10 distort the I/Q trace of the received ultrasound signal and result in irregular phase variation. To address such variation, controller 12 first divides the I/Q traces into short segments, during each of which apparatus 10 can be assumed as motionless. Then, controller 12 approximates each segment back to the closest circular arc on the complex plane. The phase variation can then be corrected by normalizing the centers of all the arcs back to the origin on the complex plane, making the I/Q trace to a collection of concentric arcs, such as shown in FIG. 6A.

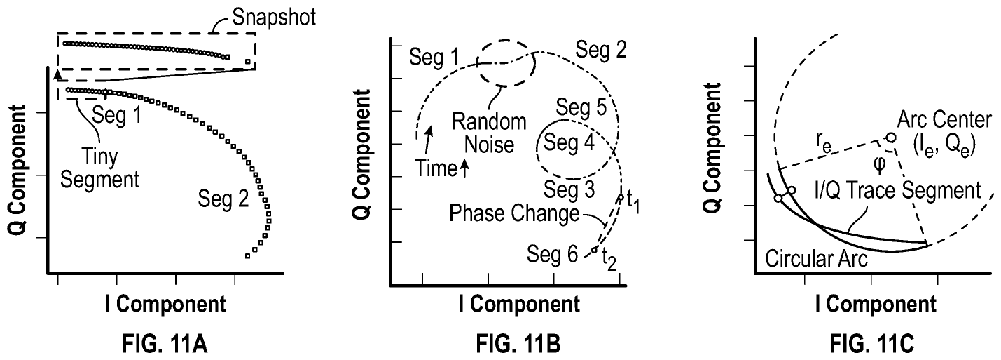

Segmentation: One intuitive method is to divide the I/Q trace into segments with the equal number of signal samples, but such approach is ineffective when chest motion is measured as a phase change: as shown in FIG. 11A, since each sample [I(t), Q(t)] has a phase of tan⁻¹(Q(t)/I(t)), smaller chest motion results in many consecutive samples with similar phases, and creates many unwanted tiny segments. Instead, controller 12 segments the I/Q trace based on its specific phase change over time. As shown in FIG. 11B, for every two consecutive samples at time t1 and t2, we compute the phase change as (Q(t2)−Q(t1))/(I(t2)−I(t1)), and produce a new segment once the cumulative phase change exceeds a threshold. For example, when this threshold is π/2, the I/Q trace in FIG. 11B is divided into 6 segments. In practice, controller 12 adaptively adjusts this threshold, to make sure that each segment contains a sufficient number of signal samples for correcting the signal distortions. The segment 2 in FIG. 11B, as an instance, corresponds to fast chest motion and hence a larger threshold of n is being applied.

Random signal noise may be produced by the hardware imperfection of smartphones or surrounding signal sources (e.g., spinning fans), and temporarily fluctuates the signal phase as shown in FIG. 11B. Such phase fluctuation may result in small unwanted segments, but unfortunately cannot be removed by smoothing the I/Q trace with a sliding window, due to the uneven distribution of signal samples such as shown in FIG. 11A. Instead, such unwanted segments are avoided by setting up a threshold on the minimum segment length. This threshold is empirically set as three times the standard deviation of I/Q samples during full inhalation in spirometry, where the chest wall motion is considered as minimum. As shown in FIG. 11C, each segment is approximated to the closet circular arc, indicated by the best linear unbiased estimates of arc center (I_{c}, Q_{c}) and radius r_{c}. This arc is estimated as:

[Î_{c},\hat{Q}_{c},\hat{θ}]^{T}=(H^{T}H)⁻¹H^{T}Y

where,

$$H = [ \begin{matrix}2 I ( 1 ) & 2 Q ( 1 ) & 1 \\ 2 I ( 2 ) & 2 Q ( 2 ) & 1 \\ ⋮ & ⋮ & ⋮ \\ 2 I ( N ) & 2 Q ( N ) & 1\end{matrix} ] , \\ Y = [ \begin{matrix}{I ( 1 )}^{2} + {Q ( 1 )}^{2} \\ {I ( 2 )}^{2} + {Q ( 2 )}^{2} \\ ⋮ \\ {I ( N )}^{2} + {Q ( N )}^{2}\end{matrix} ] ,$$

N is the segment's number of samples, and θ=p_{c}²−Î_{c}²−\hat{Q}_{c}².

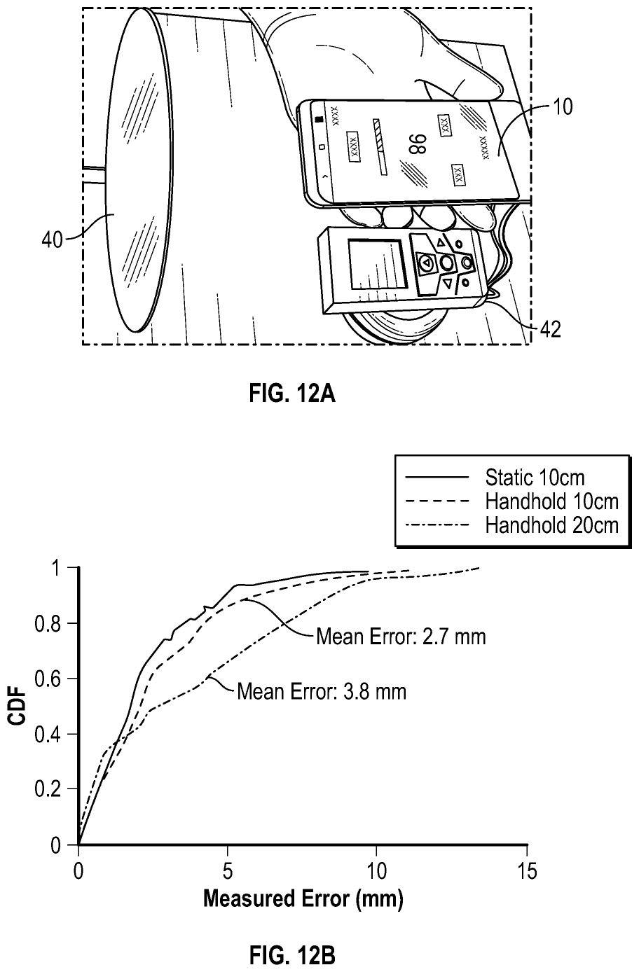

Each sample is then mapped to the arc individually. The effectiveness of such correction is evaluated, as shown in FIG. 12A, by tracking the motion of a round paper plate 40 and using a laser distance meter 42 as the ground truth. Results in FIG. 12B show that, when the paper plate moves back and forth at different distances, the motion tracking error of apparatus 10 approximates to that of a stationary apparatus 10, and is capped at 3.8 mm, which meets the requirements for accurate lung function estimation as previously discussed.

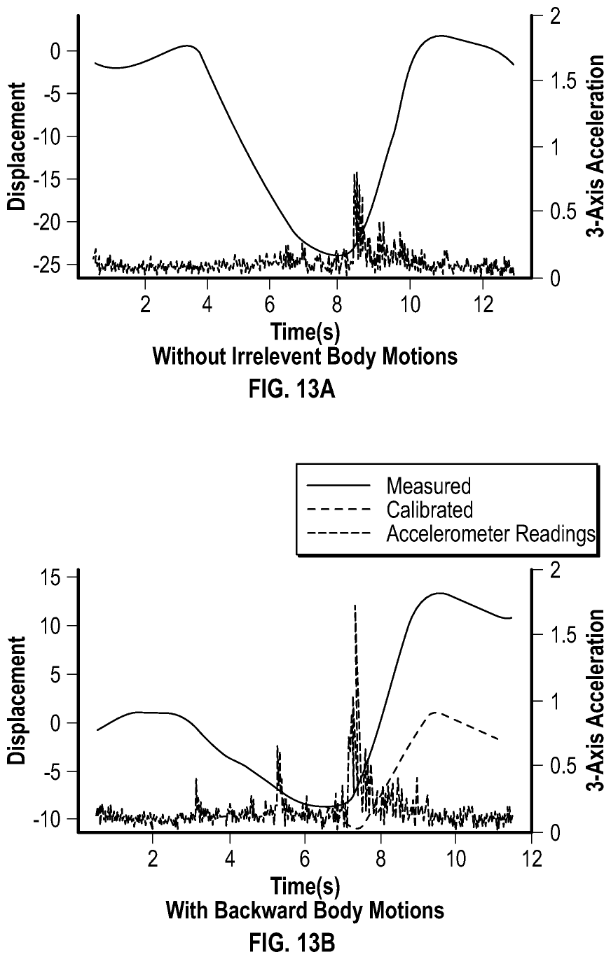

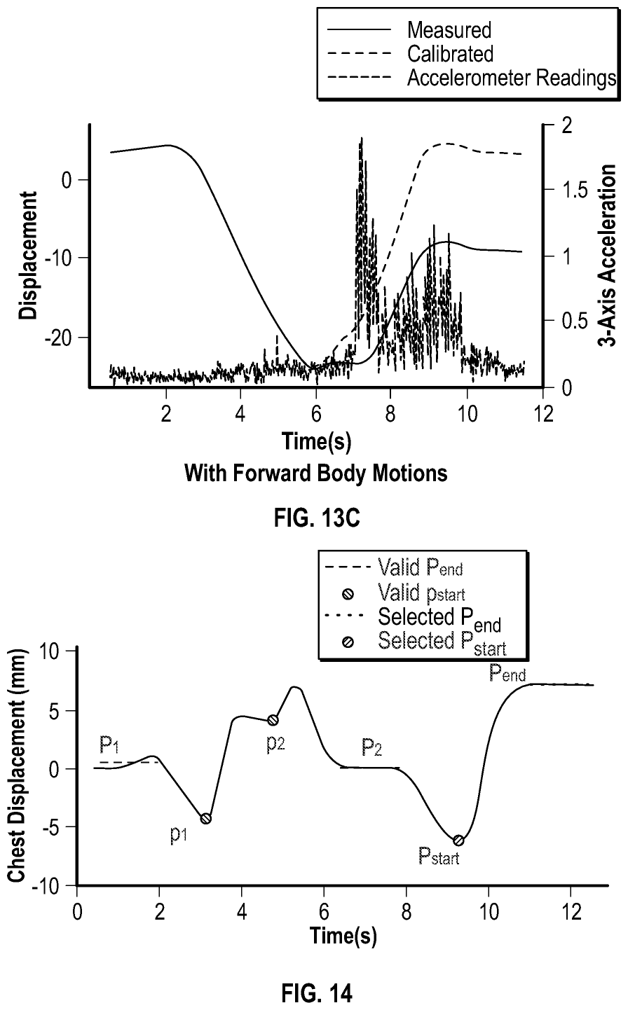

The calibration builds on the fact that the patient's chest wall should be at the same position before and after a spirometry test if there is no extra body motion during the test, because the patient is supposed to have full inhalation and exhalation in spirometry. To verify this, a digital accelerometer was attached to the patients' upper body to measure their body motions in spirometry, and the results shown in FIG. 13A match our expectation. In contrast, with noticeable body motions being indicated by the high accelerometer readings during exhalation, the patients' chest wall position will be largely changed after spirometry tests, as shown in FIGS. 13B and 13C. In practice, such body motions could be either unidirectional or bidirectional during exhalation. For unidirectional (i.e., either moving forward or backward) motions, our approach to removing its impact, as described in Algorithm 1 below, uses the chest wall position before spirometry as the baseline, to proportionally calibrate every signal sample after the exhalation starts. The outcomes, as shown in FIGS. 13B and 13C, effectively remove the difference of chest wall positions before and after spirometry.

Algorithm 1 Calibration against body movements

```
Input: D(t): the received ultrasound signal, t = 1 . . . T.
    ΔD = D_{after} − D_{before}: difference of chest wall position
Output: D′(t): the calibrated chest wall motion, t = 1 . . . T
  1: Initialize tₛₜₐᵣₜ ← exhaling starts, t_{end} ← exhaling ends:
  2: Δd ← ΔD/(t_{end} − tₛₜₐᵣₜ)
  3: for tₛₜₐᵣₜ < t ≤ t_{end} do
  $\text{4:} {D}^{′} ( t ) ← D ( t ) - ΔD \times \frac{t - {t}_{start}}{{t}_{end} - {t}_{start}}$
  5: for t_{end} < t ≤ T do
  6:  D′(t) ← D(t) − ΔD
```

In some other cases, the patient's body may move both back and forth during exhalation. When such bidirectional body motion is small, controller 12 removes this motion through adaptive smoothing: it adapts the smoothing window (W) to the momentary chest motion speed (S) as W=(1−|S/Smax|)·fs, where Smax is the maximum chest motion speed and fs is the ultrasound signal's sampling rate. In this way, slower motion leads to a larger window that produces a smoother motion curve. Rapid motion results in a smaller window to avoid missing details in the motion pattern. Big bidirectional body motions, on the other hand, indicate that the patient does not follow the spirometry protocol and controller 12 will instead judge the corresponding spirometry test as invalid. Details of such judgment are described immediately below and an evaluation of the effectiveness of such body motion removal is discussed further below.

The exhalation stage in a spirometry test is indicated by a starting point (pₛₜₐᵣₜ) and an ending plateau (P_{end}). A valid pₛₜₐᵣₜ should be a local minimum on the curve of chest wall displacement, and a valid P_{end} should correspond to a period of sufficiently small chest motion. However in practice, as shown in FIG. 14, there may be multiple possibilities of such starts and ending plateaus due to the heterogeneous human factors. To decide the best choices of pₛₜₐᵣₜ and P_{end} the following three criteria are used:
pₛₜₐᵣₜ and P_{end} are always decided in pairs, and the pₛₜₐᵣₜ always locates before P_{end}.
The average chest wall displacement within P_{end} should be higher than 90% of the maximum displacement between pₛₜₐᵣₜ and P_{end}.
If multiple pairs are available, the pair that corresponds to the maximum chest displacement in exhalation is selected.

Based on such decision, the motion features Sₘₐₓ, D₁ₛ and Dₘₐₓ, such as shown and previously discussed in regard to FIG. 8, can be calculated. If a valid pair of pₛₜₐᵣₜ and P_{end} cannot be found, it is considered that the patient did not fully follow the spirometry protocol (e.g., the body moves back and forth during exhalation), and data in this spirometry test has low quality. Such data is thus excluded from lung function estimation.

FIGS. 15A-15C show example displays of an embodiment of the present invention wherein apparatus 10 is a smartphone and the methodology described herein is implemented by the controller 12 thereof as an Android smartphone app that transmits multi-tone ultrasound signals. It uses 12 ultrasound frequencies ranging from 17 kHz to 22.5 kHz with 0.5 kHz interval, which have been proven to have good frequency responses on commodity smartphones. Being similar with previous work (e.g., such as described in Yu-Chih Tung, Duc Bui, and Kang G Shin. 2018, “Cross-platform support for rapid development of mobile acoustic sensing applications”, Proceedings of the 16th Annual International Conference on Mobile Systems, Applications, and Services, pp 455-467, the contents of which are incorporated herein by reference) the smartphone's Automatic Gain Control (AGC) was disabled to avoid unwanted fluctuations of the received signal amplitude when the ambient noise level varies. The measured chest wall motion is also adaptively smoothed before any feature extraction, using a flexible sliding window.

The app having the displays shown in FIGS. 15A-15C was designed following instructions from pulmonary clinicians, to minimize the patients' cognitive and operational barriers when using apparatus 10 and the app out of clinic. As generally shown in FIG. 15B, before a spirometry test starts, a tutorial with both texts and video is provided to demonstrate the protocol. In such example, a spirometry test using apparatus 10 is: 1) fully automated such that no manual inputs (e.g., indicating the start and end of inhalation and exhalation stages) are needed from the patient; and 2) fully customizable so that the patient can opt to pause or resume the ultrasound recording at any time, such as generally shown in FIG. 15C. Uploading of the test results is also allowed so that the app can also be used in clinical studies.

Further evaluations of the example embodiment of the present invention previously discussed can be found in “SpiroSonic: Monitoring Human Lung Function via Acoustic Sensing on Commodity Smartphones” MobiCom 2020—The 26^{th} Annual International Conference on Mobile Computing and Networking, 21-25 Sep. 2020. London, United Kingdom, the contents of which are incorporated herein by reference.

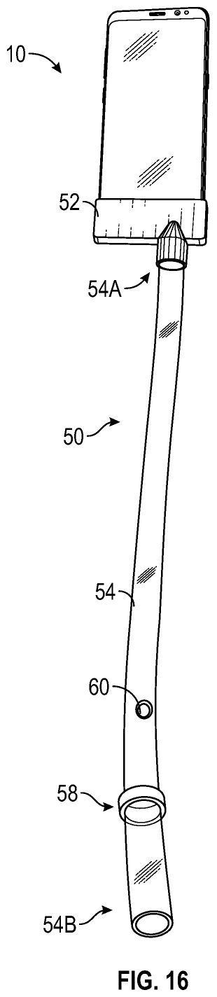

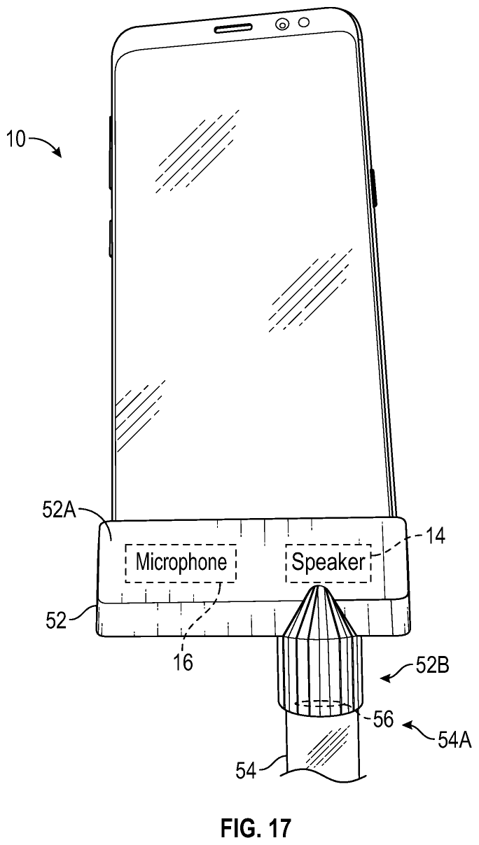

Referring now to FIGS. 16 and 17, a second arrangement in accordance with the present invention is shown in which apparatus 10, again shown as a smartphone device, is employed to provide contact-less monitoring of patient lung-function, which can serve for telemedicine and to aid in the monitoring of lung function and in the diagnosis and management of lung disease. In such arrangement, an interface 50 is employed for interfacing between apparatus 10 and a patient. Interface 50 includes an adaptor 52 and a patient interface tube 54. Adaptor 52 includes a first portion 52A that is sized and configured to engage on and around an end of apparatus 10 so as to encompass speaker 14 and microphone 16 thereof (such as shown in hidden line in FIG. 17), and a second portion 52B that is sized and configured to be coupled to a first end 54A of interface tube 54. Second portion 52B of adaptor 52 includes an opening 56 which provides for fluid communication between the interior of patient interface tube 54 and each of speaker 14 and microphone 16 of apparatus 10. Adaptor 52 may be formed from plastic or other suitable material and may be provided with any suitable mechanism or arrangement for selectively coupling with a portion of apparatus 10 so as to generally remain coupled with apparatus 10 until pulled apart from apparatus 10 with minor force (e.g., similar to the force needed to remove an average smartphone case). Interface tube 54 includes a second end 54B opposite first end 54A that is sized and configured to be placed and held in a patient's mouth as described below. To assist in such positioning within the patient's mouth, interface tube 54 further includes a stopper portion 58 for positioning second end 54B at a suitable distance inward from the front teeth of the patient. In the example shown in FIG. 16, stopper portion 58 is a ring member extending outward about interface tube 54 a selected distance inward from second end 54B that is positioned and structured for the patient to position against the outside of one or more of their front teeth. In addition, since the inner space of the interface tube 54 is otherwise sealed, an extra opening 60 is defined in interface tube 54 to ensure that the user can breathe during the test. In the example shown in FIGS. 16 and 17 interface tube 54 is formed from a clear flexible tube, however it is to be appreciated that interface tube 54 may be formed from other suitable materials and/or may be formed integrally with adaptor 52 without varying from the scope of the present invention.

As noted above, smartphone speakers emit low-intensity ultrasonic signals at 17-25 kHz, which are imperceptible to the human ear and have negligible penetration into human tissues. The disclosed concept in this aspect directs the ultrasonic signal into the user's airway via interface 50, and more particularly via adaptor 52, which is coupled to apparatus 10 as shown in FIGS. 16 and 17, and interface tube 54 with its second end 54B positioned in the user/patient's mouth. The ultrasound waveforms produced by speaker 14 are then reflected back by the airway lumens of the user and sampled by the phone's microphone 16. After filtering and denoising using advanced analytical algorithms, the disclosed concepts can isolate the mixture of reflected signals from the airway “tree” and then calculate airway mechanics, detect obstructions, evaluate airway caliber and patency, and assess measures of pulmonary function. For example, upper airway obstructions can be detected by measuring the abnormal narrowing of the airway calibers. This technology could have a broad range of clinical applications, from an early detection of changes in respiratory status to telemedicine assessment of lung function. Adaptor 52 may also include an auxiliary speaker and/or an auxiliary microphone in communication with controller 12 in order to amplify the ultrasonic signals emitted by speaker 14 of apparatus 10 and/or to further enhance the ability to capture reflected signals.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A method of estimating a number of lung function indices of an individual, comprising:
transmitting an ultrasound signal toward a chest of the individual from a speaker of a smartphone while the individual is holding the smartphone in a hand of the individual;
receiving in a microphone of the smartphone a reflected signal reflected from the chest of the individual in response to the ultrasound signal;
extracting a number of features from the reflected signal; and
providing the number of features to a neural network regression model, wherein the neural network regression model estimates the number of lung function indices based on the number of features and based on a non-linear correlation between chest wall motion and human lung function.

2. The method of claim 1, wherein the number of features are extracted from a number of exhalation portions identified from the reflected signal.

3. The method of claim 1, wherein extracting the number of features from the reflected signal comprises:
converting the reflected signal to a number of I/Q traces on a complex plane to quantify an impact of random motion of the smartphone on the reflected signal; and
adaptively removing the impact by correcting for distortion caused by the random motion and calibrating the reflected signal as if it was produced by a stationary device.

4. A non-transitory computer readable medium storing one or more programs, including instructions, which when executed by a processor, causes the processor to perform the method of claim 1.

5. An apparatus for estimating a number of lung function indices of an individual, the apparatus comprising:
a speaker;
a microphone; and
a controller coupled to the speaker and the microphone and structured and configured for:
transmitting an ultrasound signal toward a chest of the individual from the speaker while the individual is holding the apparatus in a hand of the individual;
receiving in the microphone a reflected signal reflected from the chest of the individual in response to the ultrasound signal;
extracting a number of features from the reflected signal; and
providing the number of features to a neural network regression model implemented in the apparatus, wherein the neural network regression model estimates the number of lung function indices based on the number of features and a non-linear correlation between chest wall motion and human lung function.

6. The apparatus of claim 5, wherein the number of features are extracted from a number of exhalation portions identified from the reflected signal.

7. The apparatus of claim 5, wherein extracting the number of features from the reflected signal comprises:
converting the reflected signal to a number of I/Q traces on a complex plane to quantify an impact of random motion of the smartphone on the reflected signal, and
adaptively removing the impact by correcting for distortion caused by the random motion and calibrating the reflected signal as if it was produced by a stationary device.

8. The apparatus of claim 5, wherein the apparatus is a smartphone.

9. A method of calculating airway mechanics and/or detecting airway obstructions in an individual, comprising:
directing an ultrasound signal generated by a smartphone into the airways of the individual through an interface attached to the smartphone and coupled to a speaker of the smartphone, wherein the interface has a mouthpiece structured to be received in a mouth of the individual;
receiving in a microphone of the smartphone and through the interface a reflected signal reflected from the airways of the individual in response to the ultrasound signal;
filtering and denoising the reflected signal to produce an adjusted reflected signal; and
isolating a mixture of signals in the adjusted reflected signal and calculating from the isolated mixture of signals the airway mechanics and/or detecting from the isolated mixture of signals the airway obstructions.

10. A non-transitory computer readable medium storing one or more programs, including instructions, which when executed by a processor, causes the processor to perform the method of claim 9.

11. An apparatus for calculating airway mechanics and/or detecting airway obstructions in an individual, the apparatus comprising:
a speaker;
a microphone;
an interface sized and structured to convey an ultrasound signal between the speaker and the microphone and a mouth of the individual; and
a controller coupled to the speaker and the microphone and structured and configured for:
transmitting an ultrasound signal from the speaker into the airways of the individual through the interface;
receiving from the microphone a reflected signal from the interface reflected from the airways of the individual in response to the ultrasound signal;
filtering and denoising the reflected signal to produce an adjusted reflected signal; and
isolating a mixture of signals in the adjusted reflected signal and calculating from the isolated mixture of signals the airway mechanics and/or detecting from the isolated mixture of signals the airway obstructions.

12. The apparatus of claim 11, wherein the apparatus is a smartphone.

13. The apparatus of claim 12, wherein the interface comprises:
an interface tube having a first end and a second end opposite the first end, the second end being sized and configured to be placed in the mouth of the individual; and
an adaptor having a first portion selectively engaged on and around an end of the smartphone so as to encompass the speaker and the microphone and a second portion coupled to the first end of the interface tube.

14. The apparatus of claim 13, wherein the interface tube further includes a stopper portion structured to position the second end of the interface tube a predetermined distance from a front tooth of the individual.

15. The apparatus of claim 13, wherein the interface tube includes an opening defined therein between the first end and the second end thereof.

16. The apparatus of claim 11, wherein the adaptor comprises one or more of an auxiliary speaker and/or an auxiliary microphone in communication with the controller.
